(19) United States
(12) Patent Application Publication
(10) Pub. No.: US 2014/0107321 A1
(43) Pub. Date: Apr. 17, 2014

(54) PURIFICATION OF ANTIBODIES

(71) Applicants: Kishore Jahagirdar, Hyderabad (IN); Neeru Gupta, Ambala (IN)

(72) Inventors: Kishore Jahagirdar, Hyderabad (IN); Neeru Gupta, Ambala (IN)

(73) Assignee: DR. REDDY'S LABORATORIES LIMITED, Qutubullapur (IN)

(21) Appl. No.: 14/118,813

(22) PCT Filed: May 24, 2012
(86) PCT No.: PCT/IB2012/052594

§ 371 Date: Nov. 19, 2013

(30) Foreign Application Priority Data
May 26, 2011 (IN) 1783/CHE/2011

(51) Int. Cl. C07K 1/16

(52) U.S. Cl. CPC C07K 1/165
USPC 530/387.1

(57) ABSTRACT

The invention provides a method of purification of antibodies using chromatographic technique. The method involves the use of cation-exchange chromatography for the purification of the antibody. The purified antibody can be used as a therapeutic composition.

### FIELD OF THE INVENTION

The present invention relates to a method of purification of antibodies comprising a cation exchange chromatography step.

### BACKGROUND OF THE INVENTION

Large-scale purification of proteins remains a significant challenge in the biopharmaceutical industry as efficient and cost-effective methods are required to achieve desired yields and purity levels. Therapeutic proteins are primarily products of recombinant DNA technology, i.e., cloning and expression of a heterologus gene in prokaryotic or eukaryotic systems. However, proteins expressed by recombinant DNA methods are typically associated with impurities such as host cell proteins (HCP), host cell DNA (HCD), viruses, etc. Also, there is significant heterogeneity in the expression of the desired protein, in the form of charged variants (typically acidic, lower pl variants and basic, higher pl variants). Further, multimeric proteins, such as antibodies, have a higher tendency to aggregate, contributing to significantly increased impurity levels.

The presence of these impurities, including aggregates and undesirable charged variants, is a potential health risk, and hence their removal from a final product is a regulatory requirement. Thus drug regulatory agencies such as United States Food and Drug administration (FDA) require that biopharmaceuticals be free from impurities, both product related (aggregates or degradation products) and process related (media components, HCP, DNA, chromatographic media used in purification, endotoxins, viruses, etc). See, Office of Biologics Research and Review, Food and Drug Administration, Points to consider in the production and testing of new drugs and biologicals produced by recombinant DNA technology (Draft), 1985. Thus, elimination of impurities from a final product is mandatory and poses a significant challenge in the development of methods for the purification of therapeutic proteins.

Antibodies constitute one of the most important classes of therapeutic proteins, especially in the areas of oncology, arthritis and other chronic diseases. The prior art discloses various methods for purification of crude or partially purified antibodies. WO 89/05157 teaches purification of immunoglobulins by cation-exchange chromatography using varying pH and salt concentrations in the wash and elution steps. WO 2004/024866 describes a method of purifying a polypeptide by ion exchange chromatography in which a gradient wash with differing salt concentrations is used to resolve the polypeptide. WO 1999/057134 describes the use of ion exchange chromatography for purification of polypeptides by varying conductivity and/or pH. U.S. Pat. No. 5,110,913 claims purification of murine antibodies using low pH and at least three different pH conditions in the ion-exchange chromatographic step. However the prior art typically describe the use of low pH wash conditions that may be accompanied by frequent and significant change in pH conditions during load/wash/elution steps. Such frequent alterations of pH conditions during chromatography may result in considerable reduction of antibody yield and, further, may decrease the stability of the antibody. Moreover frequent pH changes during chromatography pose difficulties in scale up.

Given the cumbersome nature of the conditions described in the prior art, alternative methods for purification of antibodies that alleviate some of the difficulties described above is desirable. The principle object of the present invention is to provide an improved method for obtaining antibody preparations that avoids use of low pH conditions and significantly reduces alterations in pH during a chromatographic step resulting in effective separation of charge variants, increased purity and recovery of the desired antibody.

### SUMMARY OF THE INVENTION

The present invention describes a process for the purification of antibodies from a mixture of impurities using cation exchange chromatography wherein the pH of a first wash buffer is similar to the pH of the load buffer, and pH of a second wash buffer is similar to the pH of the elution buffer.

### BRIEF DESCRIPTION OF THE DRAWINGS

FIG. 1 is an illustration of a chromatogram from the procedure as described in Example 1. The line marked “Cond” represents the increase in conductivity in mS/cm. Peak A, represents the eluate obtained from protein A chromatography resin.

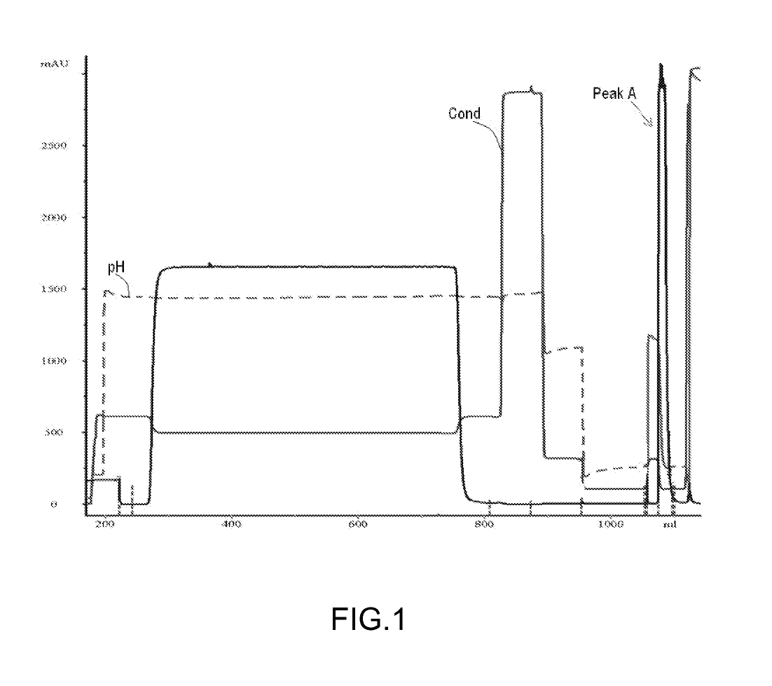

FIG. 2 is an illustration of a chromatogram from the procedure as described in Example 2. Peak A and B represent the eluate obtained from cation exchange chromatography. Peak A and B are charge variants of the anti-CD20 antibody.

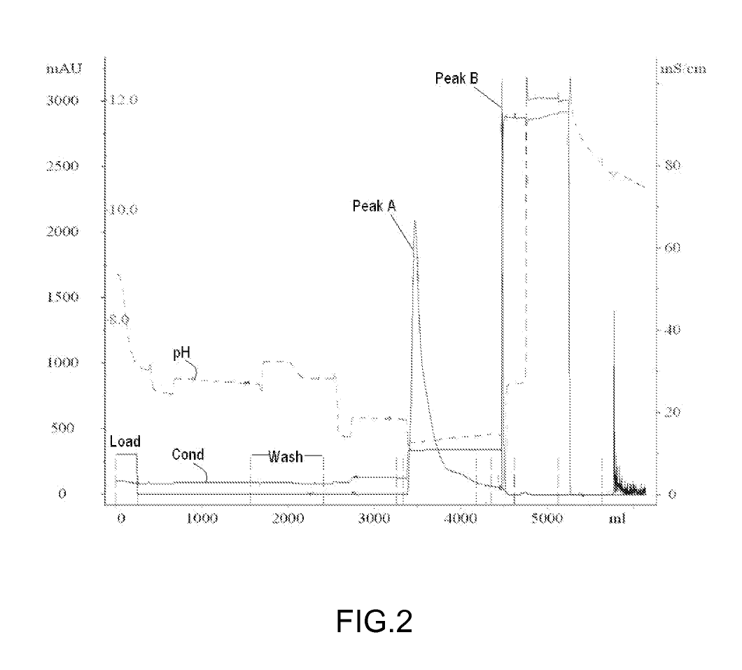

FIG. 3 is an illustration of a chromatogram from the procedure as described in Example 2. The consistency of elution in multiple runs is shown.

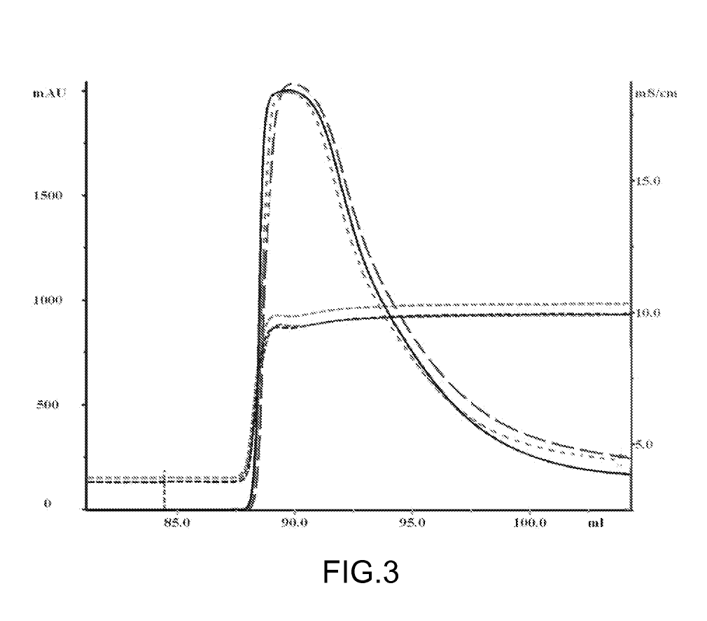

FIG. 4 is an illustration of a chromatogram from the procedure as described in Example 3. Figure represents the flow-through fraction of the anion exchange chromatography.

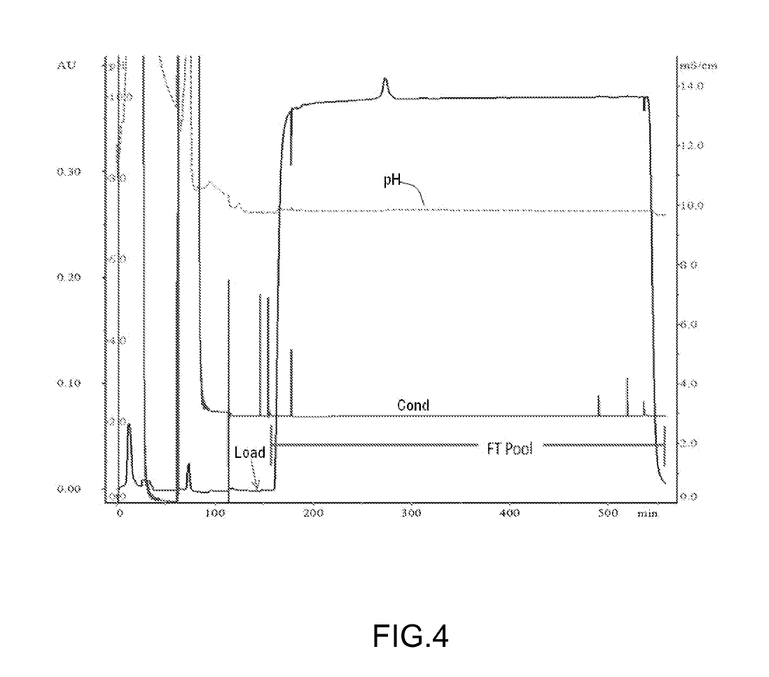

### DETAILED DESCRIPTION OF THE INVENTION

The present invention describes a process for purification of antibodies by cation exchange chromatography. The chromatographic conditions require minimal pH adjustments during load/wash/elution steps. The process described in the present invention also avoids use of low pH buffers. The conditions described in the present invention results in effective separation of charged variants, as well as removal of impurities such as HCP, aggregates and Protein A leachates and an optimum yield of the desired antibody.

The term “antibody” as used herein refers to immunoglobulins and can be isolated from various sources, such as murine, human, recombinant etc. In its broadest sense it includes monoclonal antibodies, polyclonal antibodies, multispecific antibodies and antibody fragments. It also includes truncated antibodies, chimeric, humanized or pegylated antibodies, isotypes, allotypes and alleles of immunoglobulin genes and fusion proteins, which contain an immunoglobulin moiety.

The term “impurities” as used herein refers to a material that is different from the desired polypeptide. They may be nucleic acids such as host cell DNA, host cell proteins, variants of the desired polypeptide, another polypeptide, endotoxin etc.

The term “low pH” as used herein refers to a pH of less than 6.0

The term “load buffer” as used herein refers to the buffer that is used to load the composition comprising the antibody of interest and one or more impurities onto the ion exchange support.

The term “wash buffer” as used herein refers to a buffer that is used to wash or re-equilibrate the ion exchange support, or to elute one or more impurities from the ion exchange support, prior to elution of the antibody of interest.

The “elution buffer” is used to elute the antibody of interest from the ion exchange support.

In an embodiment, the invention provides a method of the purification of antibodies comprising,
a) Loading the antibody containing solution onto a cation exchange support at a particular pH
b) Washing the support with a first wash buffer at a pH similar to the pH of the load buffer
c) Washing the support with a second wash buffer at a second pH
d) Eluting the antibody from the support using an elution buffer at a pH similar to the second wash buffer,
   wherein the pH of the two wash buffers are greater than 6.

In an embodiment, the pH of the second wash buffer is less than the pH of the first wash buffer.

In another embodiment, the invention provides a method for the purification of antibodies comprising,
a) Loading the antibody containing solution onto a cation exchange support with a buffer of pH value from about pH 6.0 to about pH 8.0
b) Washing the support with a wash buffer of pH value from about pH 6.0 to about pH 8.0
c) Washing the support with a second wash buffer of pH about 6.5
d) Eluting the antibody from the support using an elution buffer of pH about 6.5

In an embodiment, a protein-A chromatography, may precede the cation exchange chromatography.

In an embodiment, another ion-exchange chromatography or a hydrophobic interaction chromatography may precede or follow the cation exchange chromatography.

The embodiments mentioned herein may optionally include one or more tangential flow filtration, concentration, diafiltration or ultra filtration steps.

The embodiments mentioned herein optionally include one or more viral inactivation steps or sterile filtration or nano filtration steps.

The embodiments mentioned herein may include one or more neutralization steps.

The protein A chromatographic resin used may be any protein A or variant or a functional fragment thereof coupled to any chromatographic support. In embodiments, the protein A resin is Mabselect™ (GE-Healthcare Life sciences), an affinity matrix with recombinant Protein-A ligand. The resin is made of highly cross-linked agarose matrix.

Cation exchange chromatographic step mentioned in the embodiments may be carried out using any weak or strong cation exchange chromatographic resin or a membrane, which could function as a weak or a strong cation exchanger. Commercially available cation exchange support include a resin, but are not limited to, those having a sulfonate based group e.g., MonoS, MiniS, Source 15S and 30S, SP Sepharose Fast Flow, SP Sepharose High Performance from GE Healthcare, Toyopearl SP-650S and SP-650M from Tosoh, S-Ceramic Hyper D, from Pall Corporation or a carboxymethyl based group e.g., CM Sepharose Fast Flow from GE Healthcare, Macro-Prep CM from BioRad, CM-Ceramic Hyper D, from Pall Corporation, Toyopearl CM-650S, CM-650M and CM-650C from Tosoh. Alternatively, the support could be a monolithic column, disk or tubular, that performs the function of a cation exchanger. In embodiments of the invention, a strong cation exchange resin, such as SP-Sepharose® (GE Healthcare Life Sciences) is used. This resin is made using a highly cross-linked, 6% agarose matrix attached to a sulfopropyl functional group.

Anion exchange chromatography mentioned in the embodiments may be carried out using any weak or strong anion exchange chromatographic resin or a membrane which could function as a weak or a strong anion exchanger. Commercially available anion exchange resins include, but are not limited to, DEAE cellulose, Poros PI 20, PI 50, HQ 10, HQ 20, HQ 50, D 50 from Applied Biosystems, MonoQ, MiniQ, Source 15Q and 3OQ, Q, DEAE and ANX Sepharose Fast Flow, Q Sepharose high Performance, QAE SEPHADEX and FAST Q SEPHAROSE from GE Healthcare, Macro-Prep DEAE and Macro-Prep High Q from Biorad, Q-Ceramic Hyper D, DEAE-Ceramic Hyper D, from Pall Corporation. In embodiments of the invention, a strong anion exchange resin, such as Q-Sepharose Fast Flow® (GE Healthcare Life Sciences) is used. This resin is made of highly cross-linked, 6% agarose matrix attached to —O—CH₂CHOHCH₂OCH₂CHOHCH₂N⁺(CH₃)₃ functional group.

Examples of buffering agents used in the buffer solutions include, but are not limited to, TRIS, phosphate, citrate, acetate, succinate, MES, MOPS, or ammonium and their salts or derivatives thereof.

The invention is more fully understood by reference to the following examples. These examples should not, however, be construed as limiting the scope of the invention.

### EXAMPLE 1

### Protein A Chromatography

An anti-CD20 antibody was cloned and expressed in a CHO cell line as described in U.S. Pat. No. 7,381,560, which is incorporated herein by reference. The cell culture broth containing the expressed antibody was harvested, clarified and subjected to protein A affinity chromatography as described below.

The clarified cell culture broth was loaded onto a protein A chromatography column (Mabselect, VL44x250, 205 mL) that was pre-equilibrated with Tris buffer solution (pH 7.0). The column was then washed with equilibration buffer. This was followed by a wash with Tris buffer (pH 7.0) with higher conductivity and a final wash with citrate buffer at pH 5. The bound antibody was eluted using citrate buffer, pH 2.5-3.5.

### EXAMPLE 2

### Cation Exchange Chromatography

The eluate obtained from the protein A chromatography procedure described in Example 1 was loaded onto a cation exchange resin (SP Sepharose, VL44x250, 304 mL) pre-equilibrated with Tris buffer (pH 7.5) at a conductivity of 3.0 to 6.0 mS/cm. This was followed by washing the resin with a wash buffer of Tris buffer (pH 7.5) at a conductivity of 3.0 to 6.0 mS/cm. A second wash step was performed with wash buffer consisting of citrate buffer, pH 6.5, at a conductivity of 3.0 to 6.0. The bound antibody was eluted using a buffer of citrate buffer, pH 6.5 at conductivity between 9-12 mS/cm.

### EXAMPLE 3

### Anion Exchange Chromatography

The eluate obtained from the cation exchange chromatography procedure described in Example 2 was loaded onto an anion exchange resin (Q-Sepharose FF, VL32x250, 80 mL) pre-equilibrated with a Tris buffer (pH 7.5) equilibration buffer. This was followed by a post load wash with equilibration buffer and the load and wash flow-through was collected.

TABLE 1

| | Protein-A | Host Cell | | |
|---|---|---|---|---|
| | leachate | Protein | Aggregate | Step |
| Sample | (ng/mg) | (ng/mg) | (%) | Recovery (%) |
| Clarified cell | — | 2202 | — | 98 |
| culture broth | | | | |
| Protein A eluate | — | 4.0 | 6.33 | 86.4 |
| Cation Exchange | 0.17 | 0.3 | 0.81 | 81.1 |
| chromatography | | | | |
| eluate | | | | |
| Anion Exchange | 0.08 | BDL | 0.67 | 95.6 |
| chromatography | | | | |
| flow through | | | | |

BDL: Below Detection Limit

TABLE 2

| | | | Basic | Basic |
|---|---|---|---|---|
| | Acidic | | Variant-1 | Variant-2 |
| Sample | variants (%) | K0 (%) | (%) | (%) |
| Protein A eluate | 10.64 | 18.03 | 64.09 | 7.24 |
| Cation Exchange | 13.73 | 23.30 | 62.97 | BDL |
| chromatography | | | | |
| eluate | | | | |
| Anion Exchange | 14.03 | 23.60 | 62.37 | BDL |
| chromatography flow | | | | |
| through | | | | |

K0: Species devoid of the C-terminal lysine residue
BDL: Below Detection Limit

1. A process for purification of an antibody, comprising,
a) loading the antibody containing solution onto a cation exchange chromatography support at a particular pH
b) washing the support with a first wash buffer at a pH similar to the pH of the load buffer
c) washing the support with a second wash buffer at a second pH and
d) eluting the antibody from the support with an elution buffer at a pH similar to the second wash buffer,
wherein the pH of the two wash buffers are greater than 6.

2. A process according to claim 1, wherein the pH value of the first wash buffer is from about pH 6.0 to about pH 8.0

3. A process according to claim 2, wherein the pH value is from about pH 6.5 to about pH 7.5.

4. A process according to claim 3, wherein the pH value is about pH 7.5.

5. A process according to claim 1, wherein the pH value of the second wash buffer is about 6.5.

6. A process according to claim 1, wherein the pH value of the elution buffer is about 6.5.

7. A process according to claim 1, wherein the cation-exchange chromatography is preceded by a protein-A affinity chromatography step.

8. A process according to claim 1, wherein the cation-exchange chromatography is preceded by an anion exchange chromatography step or a hydrophobic interaction chromatography step.

9. A process according to claim 1, wherein the cation-exchange chromatography step is followed by an anion exchange chromatography step or a hydrophobic interaction chromatography step.

10. A process according to claim 1, wherein the cation-exchange chromatography step is preceded and/or followed by one or more ion exchange chromatography steps.
